(19) United States
(12) Patent Application Publication
(10) Pub. No.: US 2015/0050744 A1
(43) Pub. Date: Feb. 19, 2015

(54) DIAGNOSTIC AGENT AND DIAGNOSTIC METHOD FOR IRRITABLE BOWEL SYNDROME INDUCED BY ABNORMAL PROLIFERATION OF ENTEROBACTERIA

(71) Applicant: TRI-X BIOMEDICAL, INC., Ichikawa-shi (JP)

(72) Inventors: Masahiro Kajiwara, Niiza-shi (JP); Yoshihisa Urita, Tokyo (JP); Hideji Nonomura, Yoshinogawa-shi (JP)

(21) Appl. No.: 14/396,141

(22) PCT Filed: Apr. 19, 2013
(86) PCT No.: PCT/JP2013/061593

§ 371 Date: Oct. 22, 2014

(30) Foreign Application Priority Data
Apr. 27, 2012 (JP) 2012-102228

(51) Int. Cl. G01N 33/497; C07C 31/26

(52) U.S. Cl. CPC G01N 33/497; C07C 31/26; C07B 2200/05
USPC 436/133; 568/852; 702/19

(57) ABSTRACT

[Problem to be Solved]

Provided are a diagnostic agent for determining the onset of an irritable bowel syndrome and a method for determining the onset of an irritable bowel syndrome using the diagnostic agent.

[Solution]

The diagnostic agent is one for detecting abnormal proliferation of enterobacteria in a patient suspected to be suffering from an irritable bowel syndrome and containing a compound that is decomposed by enterobacteria and is labeled with a carbon isotope.

[Selected Drawing] None

[Technical Field]

The present invention relates to a diagnostic agent for an irritable bowel syndrome induced by abnormal proliferation of enterobacteria and a method for determining whether there is the onset of an irritable bowel syndrome using the agent.

[Background Art]

An irritable bowel syndrome (IBS) is a disease that causes stool abnormality, such as abdominal pain, diarrhea, or constipation, or exacerbates these symptoms and also shows psychogenic pathological conditions. IBS is classified into, for example, a “diarrhea type”, “constipation type”, or “alternating diarrhea and constipation type or mixed type” depending on the symptoms of a patient. IBS may also cause symptoms such as abnormal occurrence of gas, meteorism, abdominal pain, and convulsion. Stress is believed to be a cause, but IBS is recognized as an unexplained disease for many patients and is a disease having a risk of causing a vicious circle in which consultation itself creates additional stress.

There is no determinate diagnostic method for an irritable bowel syndrome, and a patient is usually diagnosed as suffering from the irritable bowel syndrome after negation of organic lesions of gastrointestinal tract cells and morphological abnormalities in internal organs through medical interviews and, for example, gastrointestinal endoscopy, MRI, X-ray, ultrasonic examination, and an immunocompetence test by a blood test. This diagnostic process is painful to the patient and also causes or promotes psychological anxiety and emphasizes the stress.

It has been known for more than 40 years that an irritable bowel syndrome develops after infection with enteritis. The irritable bowel syndrome (IBS) is the most common among all gastrointestinal diseases, and 11% to 14% of adults suffered from IBS. It has been also reported that more than a half of the patients have dyspepsia (Non-Patent Literature 1). In addition, it has been reported that 29.3% of acute enteritis patients develop IBS, and a hypothesis that abnormal proliferation of enterobacteria is a cause has emerged. Subsequently, since 2000, it has been reported that the symptoms of irritable bowel syndrome patients are relieved by administration of an antibacterial agent (Patent Literature 1). On the basis of these backgrounds, it has been believed that the abnormal proliferation of enterobacteria in the small intestine is the cause of an irritable bowel syndrome.

However, it is difficult to determine whether or not a human being is suffering from an irritable bowel syndrome, and the severity of the syndrome cannot be determined. The current situation is therefore that appropriate therapy depending on the severity is difficult to be carried out.

### CITATION LIST

### Patent Literature

[Patent Literature 1] Japanese National Publication of International Patent Application No. 2011-519952

### Non-Patent Literature

[Non-Patent Literature 1] G. Triadafilopoulos, et al., Digestive Dis. Sci., 1991, 36(1): 56-64

### SUMMARY OF INVENTION

### Technical Problem

Accordingly, it is an object of the present invention to provide a diagnostic agent for determining the onset of an irritable bowel syndrome and to provide a method for determining whether there is abnormal proliferation of enterobacteria and a method for determining whether there is the onset of an irritable bowel syndrome, using the diagnostic agent.

### SOLUTION OF PROBLEM

In order to achieve the object, the present inventors have diligently studied and have found that the object can be achieved by using a compound that is decomposed by enterobacteria and is labeled with a carbon isotope, and have accomplished the present invention.

That is, the present invention provides a diagnostic agent for detecting abnormal proliferation of enterobacteria in a patient suspected to be suffering from an irritable bowel syndrome. The diagnostic agent contains a compound that is decomposed by enterobacteria and is labeled with a carbon isotope.

In the diagnostic agent of the present invention, examples of the compound include compounds labeled with a carbon isotope ¹³C or ¹⁴C.

The diagnostic agent of the present invention is preferably an oral pharmaceutical agent.

The diagnostic agent of the present invention is preferably used in a breath test measuring ¹³CO₂ excreted in exhaled breath after the administration.

Examples of the compound include monosaccharides, disaccharides, oligosaccharides, celluloses, organic acids, and salts thereof.

The compound can be, for example, a monosaccharide selected from the group consisting of sorbitol, mannose, mannitol, rhamnose, arabinose, fucose, and xylitol; a disaccharide selected from the group consisting of lactulose, maltose, and lactose; an oligosaccharide selected from the group consisting of soybean oligosaccharide and isomaltooligosaccharide; a cellulose such as carmellose (carboxymethyl cellulose); an organic acid selected from the group consisting of sodium alginate, magnesium citrate, and 5-aminosalicylic acid.

The compound can be a saccharide labeled with a carbon isotope at the 1-position.

Another example of the compound is a compound (uniform material) having a carbon atom label ¹³C substituted for all carbon atoms of the molecule.

The present invention also provides a method for determining whether there is abnormal proliferation of enterobacteria, including a step of measuring the concentration of carbon isotope-labeled CO₂ or the ratio of carbon isotope-labeled CO₂ to ¹²CO₂ in exhaled breath collected from a subject administered with the diagnostic agent of the present invention.

The present invention also provides a method for determining whether there is the onset of an irritable bowel syndrome, including a step of measuring the concentration of carbon isotope-labeled CO₂ or the ratio of carbon isotope-labeled CO₂ to ¹²CO₂ in exhaled breath collected from a subject administered with the diagnostic agent of the present invention.

The present invention also provides a method for determining whether there is abnormal proliferation of enterobacteria, including a step of measuring the concentration of ¹³CO₂ or the ratio of ¹³CO₂ to ¹²CO₂ (¹³CO₂/¹²CO₂ ratio) in exhaled breath collected from a subject administered with the diagnostic agent of the present invention.

The present invention also provides a method for determining whether there is the onset of an irritable bowel syndrome, including a step of measuring the concentration of ¹³CO₂ or the ratio of ¹³CO₂ to ¹²CO₂ (¹³CO₂/¹²CO₂ ratio) in exhaled breath collected from a subject administered with the diagnostic agent of the present invention.

The methods described above preferably include a step of comparing the concentration of ¹³CO₂ or the ratio of ¹³CO₂ to ¹²CO₂ in exhaled breath before the administration of the diagnostic agent, or the rate of change (Δ¹³C value).

In the methods described above, the diagnostic agent is preferably administered in an amount of 10 mg to 15 g as the mass of the compound.

In the methods described above, the concentration of carbon isotope-labeled CO₂ or the ratio of carbon isotope-labeled CO₂ to ¹²CO₂ in exhaled breath is preferably measured before and within 60 minutes after the administration.

The methods described above may further include a step of calculating the area under the curve of concentration of ¹³CO₂ or ratio of ¹³CO₂ to ¹²CO₂ in exhaled breath formed by plotting the concentration of ¹³CO₂ or ratio of ¹³CO₂ to ¹²CO₂ in exhaled breath versus time and comparing the resulting area under the curve with that of a healthy subject.

### ADVANTAGEOUS EFFECTS OF INVENTION

The present invention provides a diagnostic agent for simply determining the onset of an irritable bowel syndrome induced by abnormal proliferation of enterobacteria and also provides a method for simply determining whether there is the onset of an irritable bowel syndrome.

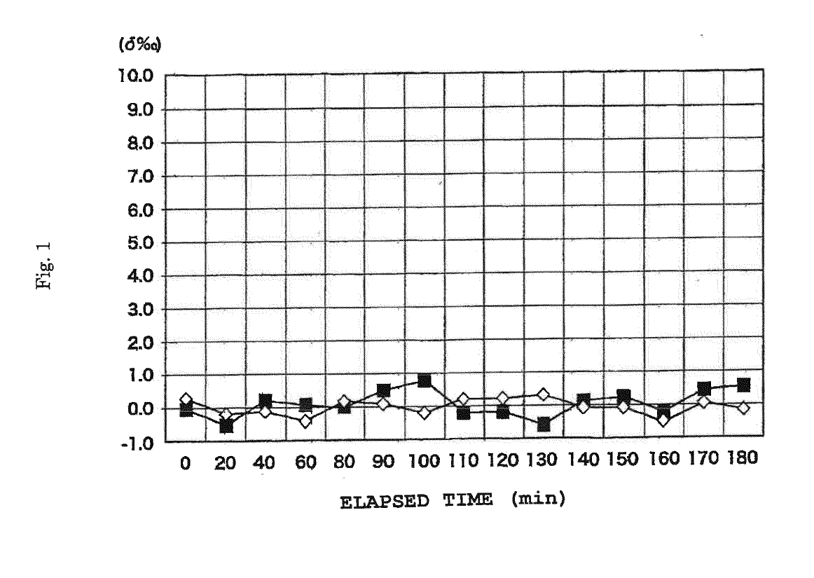

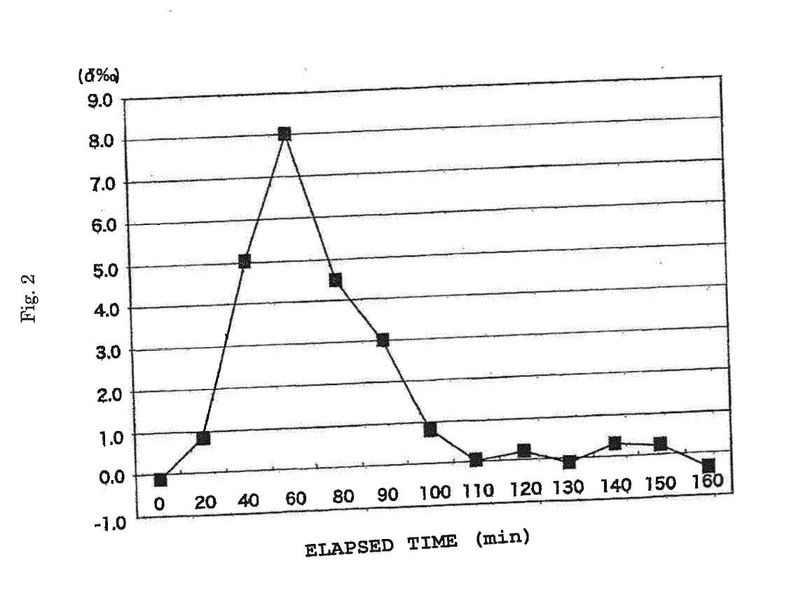

### BRIEF DESCRIPTION OF DRAWINGS

[FIG. 1] FIG. 1 is a graph showing the Δ¹³C value (‰) in exhaled breath of a healthy subject administered with ¹³C-mannitol.

[FIG. 2] FIG. 2 is a graph showing the Δ¹³C value (‰) in exhaled breath of an irritable bowel syndrome patient administered with ¹³C-mannitol.

### DESCRIPTION OF EMBODIMENTS

The irritable bowel syndrome of which the onset is determined by the present invention is classified into three types: a diarrhea type, a constipation type, and a mixture type. The diagnostic agent of the present invention is also used for determining all types of the irritable bowel syndrome, and the diagnostic agent and method of the present invention can be also used in all types of the irritable bowel syndrome. Here, the “constipation type” refers to a symptom showing a high ratio of hard feces or scybalum-like feces; the “diarrhea type” refers to a symptom showing a high ratio of soft feces or watery feces; and the “mixed type” refers to a symptom alternately showing the “constipation type” and the “diarrhea type”.

The diagnostic agent of the present invention is for detecting proliferation of enterobacteria in a patient suspected to be suffering from an irritable bowel syndrome and contains a compound that is decomposed by the enterobacteria and is labeled with a carbon isotope.

The compound contained in the diagnostic agent of the present invention is a compound labeled with a carbon isotope, and at least one carbon atom in the compound is substituted with a carbon isotope. Examples of the carbon isotope include a stable isotope ¹³C and radioactive isotopes ¹¹C and ¹⁴C. Although each isotope can be used for labeling the compound, the compound is preferably labeled with the stable isotope ¹³C because of its high safety.

The compound contained in the diagnostic agent of the present, invention is decomposed by enterobacteria, but is preferably not decomposed by human beings. Examples of such compounds include monosaccharides, disaccharides, oligosaccharides, celluloses, organic acids, and salts thereof. Examples of the monosaccharide include D-sorbitol, D-mannose, D-mannitol, L-rhamnose, L-arabinose, L-fucose, and xylitol. Examples of the disaccharide include lactose, maltose, and lactulose. Examples of the oligosaccharide include soybean oligosaccharide and isomaltooligosaccharide. Examples of the cellulose include carmellose (carboxymethyl cellulose). Examples of the organic acid include sodium alginate and magnesium citrate and also include 5-aminosalicylic acid. In addition, the compound contained in the diagnostic agent of the present invention may be a salt of the compound mentioned above. Examples of the salt include, but not limited to, mineral acid salts such as hydrochlorates and sulfates; organic acid salts such as p-toluenesulfonates; metal salts such sodium salts, potassium salts, and calcium salts; ammonium salts; organic ammonium salts such as methylammonium salts; and amino acid salts such as glycine salts.

The compound is required not to be directly decomposed and metabolized by human beings, but to be decomposed and metabolized by enterobacteria. From this viewpoint, a monosaccharide or disaccharide is preferably used. When the compound is a monosaccharide or disaccharide, the position labeled by a carbon isotope is preferably the 1-position of the saccharide. When the labeling position is the 1-position, the decomposition and metabolism by enterobacteria proceed rapidly. As a result, the diagnosis can be performed in a shorter time from the administration of the diagnostic agent.

In addition, when the compound is a monosaccharide or disaccharide, the compound may be a uniformly labeled material in which all or most of the carbon atoms of each molecule are substituted with a carbon atom label.

The compound contained in the diagnostic agent of the present invention can be synthesized by a conventionally known method. Alternatively, the compound may be a commercially available one. For example, in a case of using mannitol as the compound, ¹³C-mannitol (Cambridge Isotope Labs. USA) can be used.

As the compound contained in the diagnostic agent of the present invention, the above-mentioned compounds may be used alone. Alternatively, the diagnostic agent of the present invention may contain the above-mentioned compound or its salt in a pharmaceutical form prepared by mixing the compound or its salt with an excipient or a carrier and formulating the mixture into tablets, capsules, a powder, granules, a liquid, or the like. The diagnostic agent of the present invention is preferably administered orally and may be formulated into a dosage form suitable for oral administration by a known method in the pharmaceutical field. In a case of such formulation, the amount of the compound to be administered once can be 10 mg to 15 g. When the amount of the compound is large, such as 15 g, the compound may be divided into multiple parts. In a case of not formulating the compound, the diagnostic agent of the present invention may be dissolved in, for example, water or saline at the time of administration.

The excipient and the carrier used in formulation may be any excipient or carrier that is usually used in the art and is pharmaceutically acceptable, and the type and the composition are appropriately changed. For example, water is used as a liquid carrier. A solid carrier that can be used is, for example, a saccharide such as lactose, sucrose, or glucose; starch such as potato starch or corn starch; or a cellulose derivative such as crystalline cellulose. The diagnostic agent may further contain a lubricant such as magnesium stearate, a binder such as gelatin or hydroxypropyl cellulose, or a disintegrator such as carboxymethyl cellulose. In addition, the diagnostic agent may contain, for example, an antioxidant, a coloring agent, a corrigent, or a preserving agent. In a case of a liquid, in general, the carrier is desirably sterilized water, physiological saline, or various buffer solutions. The diagnostic agent may be used as a lyophilized formulation.

The diagnostic agent of the present invention can be used in a method for determining whether there is proliferation of enterobacteria or a method for determining whether there is the onset of an irritable bowel syndrome, as described below.

The method for determining whether there is abnormal proliferation of enterobacteria and the method for determining whether there is the onset of an irritable bowel syndrome of the present invention will now be described.

The method for determining whether there is abnormal proliferation of enterobacteria of the present invention includes a step of measuring the concentration of carbon isotope-labeled CO₂ or the ratio of carbon isotope-labeled CO₂ to ¹²CO₂ in exhaled breath collected from a subject administered with the diagnostic agent of the present invention.

As described above, whether there is proliferation of enterobacteria can be determined from a value obtained by measuring the concentration of carbon isotope-labeled CO₂ or the ratio of carbon isotope-labeled CO₂ to ¹²CO₂ in exhaled breath. In this case, the proliferation of enterobacteria can be an index allowing simultaneous determination of whether there is the onset of an irritable bowel syndrome.

The method for determining whether there is abnormal proliferation of enterobacteria and the method for determining whether there is the onset of an irritable bowel syndrome of the present invention preferably include a step of comparing the concentration of carbon isotope-labeled CO₂ or the ratio of carbon isotope-labeled CO₂ to ¹²CO₂ in exhaled breath before the administration of the diagnostic agent (calculating the Δ¹³C value). When enterobacteria abnormally proliferate, the administered diagnostic agent is decomposed by the enterobacteria, and ¹³CO₂ generated during the decomposition is transferred into blood and is then excreted in exhaled breath from the lung. As a result, the concentration of ¹³CO₂ in the exhaled breath increases, and the abnormal proliferation of the enterobacteria can be determined. Consequently, it is possible to determine that the patient is developing an irritable bowel syndrome. Therefore, the compound contained in the diagnostic agent of the present invention is preferably a compound that is not generally decomposed by human beings.

In the method for determining whether there is proliferation of enterobacteria and the method for determining whether there is the onset of an irritable bowel syndrome of the present invention, the amount of the diagnostic agent to be administered to a subject is preferably approximately 10 mg to 15 g, more preferably approximately 50 mg to 10 g, and most preferably approximately 100 mg to 1 g, as the mass of the compound contained in the diagnostic agent, and can be appropriately controlled depending on, for example, the symptoms of the subject.

That is, in the method for determining whether there is abnormal proliferation of enterobacteria and the method for determining whether there is the onset of an irritable bowel syndrome of the present invention, the diagnostic agent in a dosage form of a tablet, a capsule, a powder, granules, a liquid, or the like may be used, or the compound may be directly used in a powder form.

The exhaled breath after the administration may be collected at any time without particular limitation. For example, the concentration of ¹³CO₂ or the ratio of ¹³CO₂ to ¹²CO₂ in exhaled breath may be measured over time by collecting exhaled breath sequentially after the administration of the diagnostic agent of the present invention. However, as obvious from the results of the example described below, the behavior of the difference (Δ¹³C value: ‰) between the ¹³CO₂/¹²CO₂ concentration ratio (δ¹³C value) in exhaled breath collected at each time after the administration of the diagnostic agent and the δ¹³C value in exhaled breath before the administration of the diagnostic agent in a patient developing an irritable bowel syndrome obviously differs from that in a healthy subject. Accordingly, it is possible to determine whether there is abnormal proliferation of enterobacteria and whether there is the onset of an irritable bowel syndrome from the Δ¹³C value. The period for collecting exhaled breath is specifically within 60 minutes after the administration and may be approximately 20 to 60 minutes. Exhaled breath may be continuously collected at time intervals of, for example, 5 or 10 minutes.

The labeled CO₂ contained in exhaled breath can be measured and analyzed by a common analytical method. For example, ¹⁴CO₂ can be measured by a liquid scintillation counting method, and ¹³CO₂ can be measured by mass spectrometry, infrared spectroscopy, emission spectrometry, or magnetic resonance spectrometry. The infrared spectroscopy can use an infrared spectrometer (for example, POCone, manufactured by Otsuka Electronics Co., Ltd.). POCone is an apparatus for measuring the abundance ratio (¹³CO₂/¹²CO₂) in exhaled breath on the basis of the difference in the wavelength of infrared absorption of ¹³CO₂ and ¹²CO₂. It is preferred to further calculate the variation, Δ¹³C value (‰), from the difference from the measured value of the gas in exhaled breath before the administration showing the natural abundance ratio.

Infrared spectroscopy and mass spectrometry are preferred from the viewpoint of measurement accuracy. Oral administration of the diagnostic agent of the present invention in fasting is usually preferred.

Exhaled breath can be collected, by breathing normal exhaled breath into an exhaled breath collection bag (for example, exhaled breath collection bag exclusive for UBiT, manufactured by Otsuka Pharmaceutical Co., Ltd.).

In the method for determining whether there is abnormal proliferation of enterobacteria and the method for determining whether there is the onset of an irritable bowel syndrome of the present invention, the area under the curve of concentration of ¹³CO₂ or ratio of ¹³CO₂ to ¹²CO₂ in exhaled breath formed by plotting the concentration of ¹³CO₂ or ratio of ¹³CO₂ to ¹²CO₂ in exhaled breath versus time may be calculated and compared with the area under the curve of a healthy subject.

The area under the curve can be determined by drawing a graph by plotting the Δ¹³C values (‰) calculated from the concentration of ¹³CO₂ or the ratio of ¹³CO₂ to ¹²CO₂ in exhaled breath on the vertical axis against the elapsed time after the administration on the horizontal axis. The area under the curve of a subject is compared to that of a healthy subject. When the area under the curve of a subject is larger than that of a healthy subject, the subject is determined to have abnormal proliferation of enterobacteria or have the onset of an irritable bowel syndrome.

The method for determining whether there is proliferation of enterobacteria of the present invention is also useful for determining the therapeutic effect on an irritable bowel syndrome. That is, the therapeutic effect can be determined by measuring the concentrations of ¹³CO₂ or the ratios of ¹³CO² to ¹²CO₂ in exhaled breath before and after the start of therapy and deciding whether the concentration of ¹³CO₂ decreased or not from the measured values.

The present invention will now be described in more detail by an example, but the scope of the present invention is not limited to the following example.

### Example 1

An aqueous solution of ¹³C-mannitol was prepared by dissolving 300 mg of ¹³C-mannitol (Cambridge Isotope Labs., USA) in 100 mL of water. The aqueous solution of ¹³C-mannitol was orally administered to two healthy subjects. Exhaled breath was collected before the administration and after the administration for 180 minutes at appropriate intervals, and the concentration of ¹³CO₂ in each exhaled breath was measured with POCone (manufactured by Otsuka Electronics Co., Ltd.). The ¹³CO₂/¹²CO₂ concentration ratio (δ¹³C value) in each collected exhaled breath sample was measured, and the difference, Δ¹³C value (‰), between the δ¹³C value (‰) in exhaled breath before the administration and the δ¹³C value (‰) in exhaled breath after the administration was calculated. The results are shown in FIG. 1.

As shown in FIG. 1, in a healthy subject, the Δ¹³C value (‰) was in a range of −0.5 to 0.9 (‰) from immediately after to 180 minutes after the administration.

Similarly, an aqueous solution of ¹³C-mannitol was administered to a subject suffering from an irritable bowel syndrome, and similarly the Δ¹³C value (‰) in exhaled breath was measured. The results are shown in FIG. 2.

As shown in FIG. 2, the Δ¹³C value increased up to 5 (‰) within 20 minutes after the administration, increased up to 8 (‰) at 30 minutes alter the administration, and then decreased.

The results described, above reveal that whether there is abnormal proliferation of enterobacteria and whether there is the onset of an irritable bowel syndrome can be determined by using the diagnostic agent of the present invention.

1. A diagnostic agent for detecting abnormal proliferation of enterobacteria in a patient suspected to be suffering from an irritable bowel syndrome, the diagnostic agent comprising a compound that is decomposed by enterobacteria and is labeled with a carbon isotope ¹³C.

2. (canceled)

3. The diagnostic agent according to claim 1, wherein the diagnostic agent is an oral pharmaceutical agent.

4. The diagnostic agent according to claim 1, wherein the diagnostic agent is used in a breath test for measuring ¹³CO₂ excreted in exhaled breath after the administration.

5. The diagnostic agent according to claim 1, wherein the compound is selected from the group consisting of monosaccharides, disaccharides, oligosaccharides, celluloses, organic acids, and salts thereof.

6. The diagnostic agent according to claim 1, wherein the compound is a monosaccharide selected from the group consisting of sorbitol, mannose, mannitol, rhamnose, arabinose, fucose, and xylitol; a disaccharide selected from the group consisting of lactulose, maltose, and lactose; an oligosaccharide selected from the group consisting of soybean oligosaccharide and isomaltooligosaccharide; carmellose (carboxymethyl cellulose); or an organic acid selected from the group consisting of sodium alginate, magnesium citrate, and 5-aminosalicylic acid.

7. The diagnostic agent according to claim 5, wherein the compound is a saccharide labeled with a carbon isotope at the 1-position.

8. The diagnostic agent according to claim 1, wherein the compound has a carbon isotope ¹³C substituted for all carbon atoms of the molecule (uniform material).

9. (canceled)

10. (canceled)

11. A method for determining whether there is abnormal proliferation of enterobacteria, the method comprising:
a step of measuring the concentration of ¹³CO₂ or the ratio of ¹³CO₂ to ¹²CO₂ (¹³CO₂/¹²CO₂ ratio) in exhaled breath collected from a subject administered with the diagnostic agent according to claim 1.

12. A method for determining whether there is the onset of an irritable bowel syndrome, the method comprising:
a step of measuring the concentration of ¹³CO₂ or the ratio of ¹³CO₂ to ¹²CO₂ (¹³CO₂/¹²CO₂ ratio) in exhaled breath collected from a subject administered with the diagnostic agent according to claim 1.

13. The method according to claim 11, further comprising:
a step of comparing the concentration of ¹³CO₂ or the ratio of ¹³CO₂ to ¹²CO₂ to those in exhaled breath before the administration of the diagnostic agent.

14. The method according claim 11, wherein the diagnostic agent is administered in an amount of 10 mg to 15 g as the mass of the compound.

15. The method according to claim 11, wherein the concentration of ¹³CO₂ or the ratio of ¹³CO₂ to ¹²CO₂ in exhaled breath is measured before and within 60 minutes after the administration.

16. The method according to claim 11, the method further comprising:
a step of calculating the area under the exhaled breath curve formed by plotting the concentration of ¹³CO₂ or ratio of ¹³CO₂ to ¹²CO₂ in exhaled breath versus time and comparing the resulting area under the curve with that of a healthy subject.
